(19) United States
(12) Patent Application Publication
(10) Pub. No.: US 2024/0049972 A1
(43) Pub. Date: Feb. 15, 2024

(54) MEASUREMENT DEVICE FOR MEASURING VITAL PARAMETERS AND METHOD FOR DETERMINING VITAL PARAMETERS

(71) Applicants: Gianluca STALDER, Zürich (CH); Luisa MARKUN, Zürich (CH)

(72) Inventors: Gianluca STALDER, Zürich (CH); Luisa MARKUN, Zürich (CH)

(21) Appl. No.: 18/268,881

(22) PCT Filed: Oct. 22, 2021
(86) PCT No.: PCT/EP2021/079410

§ 371 Date: Jun. 21, 2023

(30) Foreign Application Priority Data
Dec. 23, 2020 (WO) PCT/EP2020/087794

(51) Int. Cl. A61B 5/021; A61B 5/0225; A61B 5/1495; A61B 5/00; A61B 5/145

(52) U.S. Cl. CPC A61B 5/02141; A61B 5/0225; A61B 5/1495; A61B 5/6843; A61B 5/14532; A61B 5/14542; A61B 5/14517

(57) ABSTRACT

A measurement device (1) for measuring at least one vital parameter is provided. The measurement device (1) comprises a base body (2), a fixing means (10) and at least one sensor (5). The at least one sensor (5) is mounted in a damped manner, in particular actively damped, by a means for pressure regulation (7) in the base body (2), in particular in a recess (3) of the base body (2). Furthermore, a measurement system with a measurement device (1) according to the invention, a method for determining a pressure with which the sensor (5) of the measurement device lies on the skin of the patient, a method for measuring at least one vital parameter and a use of the measurement device for determining at least one vital parameter are provided.

The present invention relates to a measurement device for measuring vital parameters. Moreover, the present invention addresses a method for determining vital parameters and a use of the measurement device for determining vital parameters.

In modern medicine, which in particular is trying to orient itself from the broad mass to a single individual, that is towards so-called “personalized medicine”, it is of especial significance to have as many single patient's metrics as possible. But also by the further technological development of smartphones and their ability to communicate with sensors and to administer the obtained data and even to evaluate them, there is a trend towards self-monitoring of vital parameters, as e.g., heart rate, blood pressure, the body temperature and respiratory rate. Measurement devices for measuring vital parameters are especially popular with athletes, since in this way they can track and optimize their training success. Another vital parameter, of which the regular monitoring stands in the foreground especially for diabetics, is the blood sugar level. To get an overview of the function and activity of the lung, the oxygen saturation in the blood is a preferably consulted vital parameter. As big as the demand for measurement devices is also the offer for such devices, and not all of them allow a reliable determination and particularly precise determination of the vital parameters. In particular, measurement devices which are worn for a longer time or even permanently on the body can provide incorrect measurement values. The reason for this is that the measurement device itself interacts with the body, for example by exerting pressure, thereby impairing the system to be measured so that not the value present without such an interaction but only the falsified, impaired measurement value can be determined. The significance of the latter is correspondingly unreliable.

The problem to be solved by the present invention is to provide a measurement device for measuring vital parameters which allows a precise and in particular less falsified determination of at least one vital parameter.

This problem is solved by a measurement device for measuring vital parameters according to claim 1.

Said measurement device comprises a base body, a fixing means and at least one sensor. The at least one sensor is characterized in that it is mounted in a damped manner, in particular actively damped, by a means for pressure regulation in the base body, in particular in a recess of the base body.

This means that the sensor is not rigidly connected to the base body, meaning in this context that the sensor can move relative to the base body, specifically related to at least one spatial direction. The connection is a movable one or elastic connection and not a solid one or in other words rigid connection. By the fact that the sensor can especially move in the spatial direction relative to the base body, which in the put-on state or worn state of the measurement device stands orthogonal to the body surface or skin of the patient, the sensor can rest on or abut the skin with a different pressure than the rest of the measurement device, in particular its main body. By the damped mounting of the sensor in the base body, external forces that act on the base body are only weakly and ideally not at all transferred to the sensor so that the external forces preferably do not have any influence on the pressure of the sensor on the skin and in this way do not falsify the measurements of the sensor. Thereby it is important that the sensor is mounted and remains in the base body (e.g. within the recess of the base body; meaning that the sensor does not protrude over the base body but that the side of the sensor directed to the skin is fully within the recess and protrudes maximally up to the end of the recess that is directed to the skin) since in this way an external force acting on the base body is transferred to the skin surface and not to the sensor. By an active damping it is possible to mostly eliminate a transfer of an external force acting on the base body to the sensor. It is to be mentioned that the fixing means also represents an external force on the base body so that it is fixed to the body in an appropriate manner. By the damped mounting of the sensor in the base body the pressure of the sensor on the skin surface is (mostly) independent of for example how strongly a fastening strap is tightened and hence to the extent the (external) force of the fastening strap acts on the base body to fasten it to the body. To be further mentioned, it is advantageous to mount only the sensor itself by the means for pressure regulation and to arrange or place all other component parts (as far as possible) in the base body to keep the mounted damped mass as small as possible. All “heavy” components should be part of the base body and should not stress the sensor. The damped mounting of the sensor in the base body allows also to (partially) compensate for the external forces such as gravity or the forces that act on the sensor because of movements of the wearer of the measurement device. So, it can also be guaranteed in particular that the sensor remains on the desired measuring position and will be not moved away from it by the external forces.

Here it should be mentioned that the term “patient” is equivalent to the human or animal of whom at least one vital parameter is to be measured. The invention is on no account to be limited to the application on humans, instead it can equally be used for the measurement of vital parameters of animals. Concurrently, the term “patient” is not to be understood here in such a narrow meaning that the said person or said animal must necessarily be in a medical/veterinary treatment. In the context of this invention “patient” rather means “the human/the animal of whom at least one vital parameter should be measured”.

The measurement device is an apparatus for continuous, non-invasive collecting of body values, for example such as O₂, blood pressure, blood glycose etc., which is attached to the body of the patient, for example to the upper arm, wrist, thigh etc. The measurement device has at least one integrated measurement range which within the scope of this invention is defined as sensor.

To better understand the meaning of the present invention, the medical background is subsequently explained:

The normal venous pressure of a human during the day is normally (at least for around 99.9% of humans) between 6 mmHg and 18 mmHg and only in exceptional cases higher or lower. A low venous pressure of about 6 mmHg is rather present in elderly patients with underlying health conditions, a high venous pressure of about 18 mmHg is rather correct for young and physically healthy patients in the normal condition, wherein the normal condition describes for example the upper arm in resting state, sitting, or standing. A higher venous pressure up to 22 mmHg or even 30 mmHg occurs under physical stress, for example while exercising of young resp. healthy body and in patients with a healthy, strong and thick skin, so patients without degenerative skin problems e.g. caused by solar radiation. A lower venous pressure up to 2 mmHg occurs in elderly patients, in patients in a physically bad condition, in patients with underlying health conditions, such as for example diabetes, in patients who are bedridden, sleep or are operated, are under medication influence etc., or in patients whose skin is sagging or for example damaged by excessive exposure to solar radiation.

To be further noted, the normal venous pressure is a venous pressure that is required to avoid a so-called decubitus. A decubitus is understood as a local damage to the skin and the underlying tissue caused by longer pressure stress which disrupts or disables the perfusion of the skin. So, a decubitus occurs when the veins are partially or fully clamped.

Nonetheless, during this pressure stress for example oxygen is consumed. As consequence, in the area affected by the decubitus there is a blood deficiency and so an undersupply of important blood components. To avoid an undersupply the sensor pressure cannot be so big that it (greatly) exceeds the normal venous pressure. An exceed of this venous pressure would result in a reduced perfusion. It means that the sensor pressure must be smaller or equal the normal venous pressure depending on the individual patient, in order not to hinder the blood flow. As soon as the blood flow is hindered or stopped, the blood values and the blood pressure in the veins change. The blood values etc. measured in such a state do not correspond to the other values in the body of the person. Not applicable or falsified measurement results are the consequence. Starting from normal venous pressures between 6 mmHg and 18 mmHg an optimal pressure of the sensor (not of the measurement device itself or its base body!) of between 2 mmHg and 30 mmHg for a human, results on the skin. If the patient is for example a bigger animal, such as for example a horse, dromedary or suchlike, the pressure of the sensor must be extended up to 60 mmHg.

The relation between blood flow and measured values can be further illustrated as follows:

When little or no blood flows, e.g. the oxygen or the blood glucose are anyways consumed under the measurement area and the composition of the blood additionally changes by the decreased flow, physically induced. As a result, the value measured under the sensor is falsified. This, depending on the activity of the patient, for example rest phase after sport, bedriddenness etc., the measured value (O₂, blood glucose etc.) can drop over several minutes far under the actual value in the rest of the body. If the respective decisive venous pressure is exceeded by the sensor in form of the sensor pressure in a person, no or less blood flows. The result is non-correct measurement values or measurement values which are not corresponding to the residual (blood) picture in the body. It arises a “pumping” of the measurement signal. If during this phase the blood glucose value was measured and, accordingly to these values, e.g. insulin was administered, or a signal, or an alarm was triggered, it would be fatal.

In a lying patient, for example during an operation or longer bedriddenness, medicinal treatment etc., the venous pressure can drop to 2-3 mmHg. The reason for this is that the venous pressure, next to other influencing factors, also depends on the distance of the heart to the measurement point, wherein the “distance” is to understand as height difference and describes the vector corresponding to the gravity. In a standing patient, the venous pressure is continuously decreasing from the leg to the heart. In turn, in a lying patient the placement position of the measurement device, irrespective if it is the arm or leg, is almost at the same height as the heart, the “distance” is quasi zero. In turn, in the universe, where there is no gravity, the venous pressure will by time settle down to about 10 mmHg, such as in a lying, healthy patient, for example at the upper arm, outside the body, not inside the body. The difference to the inside of the body is reasoned by the condition of the communicating vessels, which is present in the inside. However, on the extremities, for example at the upper arm, the external pressure (atmosphere) and the skin tension are opposed to the body pressure.

The sensor of the measurement device according to this invention is the base for all measurements of blood physiology, or blood composition, such as blood sugar, blood pressure, O₂ etc. and allows to determine the blood physiology, or the blood composition under the sensor, as also present in the rest of the body. This is the base for every correct non-invasive measurement of blood pressure, blood composition, such as for example oxygen content, blood sugar content, etc.

In an embodiment of the measurement device which can be combined with every already mentioned embodiment and every embodiment subsequently mentioned, unless in contradiction, the measurement device is characterized in that the at least one sensor is elastically mounted in the base body, in particular in the recess of the base body.

An elastic mounting in the base body is achieved e.g. by the base body having a recess for the reception of the at least one sensor. The recess can for example be described as a groove, a cavity, or material hollow. The recess can be a passage opening but also only a material hollow which, ideally, is each adapted to the geometry of the sensor to be embed inside. In the recess, the sensor can be mounted e.g., floating e.g., embed in an elastic plastic material which fills out the space between sensor and recess at least partially or fully. Ideally, the sensor is mounted freely with respect to the measurement device, or the base body to minimize the couplings from the fixing means (e.g. strap) and the base body. Even though, in general, the fixing means is an elastic strap, it can also be a clip, in particular in measurement devices for the finger. Alternatively, the measurement device can also be glued to the skin of the patient, so that the glue or glue strip represent the fixing means.

In an embodiment of the measurement device which can be combined with every already mentioned embodiment and every embodiment subsequently mentioned, unless in contradiction, the measurement device is characterized in that the level of damping, or the elasticity of the mounting, is regulable by the means for pressure regulation.

In an embodiment of the measurement device which can be combined with every already mentioned embodiment and every embodiment subsequently mentioned, unless in contradiction, the measurement device is characterized in that the damping of the means for pressure regulation is regulable depending on an external force acting on the sensor (e.g. a force of the fixing means on the base body and from the base body further on the sensor or the gravity acting on the sensor), in particular for the reduction, or elimination of an effect of the external force on the sensor. In this way it is possible to reduce, or even eliminate the transfer of external forces acting on the base body, on the sensor. A reduction of the external forces occurs in particular of at least 50%, preferably of at least 80% or even 90% up to complete elimination.

In an embodiment of the measurement device which can be combined with every already mentioned embodiment and every embodiment subsequently mentioned, unless in contradiction, the measurement device is characterized in that a pressure of the at least one sensor is adjustable by the means for pressure regulation in a measuring position, in particular automatically regulable, further in particular depending on a or the external force. In this way it is possible to keep the pressure constant on a desired value, independently from external forces acting on the base body. Also shearing forces between the sensor and the skin can very slightly clamp a vein and severely hinder or even stop the blood flow, so that subsequently the sensor gives false measurement results. Therefore, the desired pressure is typically specified and regulated in a way that the sensor can still glide over the skin surface without generating or receiving any shearing forces.

It is the goal that different pressures of the sensor, like contact pressures, are regulable with one measurement device, so that the measurement device can be used for different patients and the sensor pressure, that is the pressure with which the sensor presses on the skin and the underlying tissue of the patient, can be regulated individually at the normal venous pressure of each patient. Moreover, the regulation of the elasticity level, and with it the regulation of the sensor pressure, is interesting, when more than one sensor is built in the measurement device. Eventually it is important that the size of the sensor surface is considered for the regulation of the optimal contact pressure. However, this varies depending on the vital parameter to be determined and hence on the applied measurement method for determining this vital parameter. As measurement methods are applied e.g., non-invasive measurement methods, such as for example LED, sensor-stripe, ultrasound, capacitive, inductive etc. or also a combination of different measurement methods. Depending on the surface, the absolute contact pressure per sensor is higher or lower, if the sensors are built identically. Their contact pressure should be independent from the surface between 2 mmHg and 30 mmHg (60 mmHg for horses etc.). An exceeding or falling below the contact pressure does not result in a reliable signal, in particular seen over time and depending on the physical activity, or the physical condition of the patient. Accordingly, the sensor pressure should be optimally regulable, specifically regulable accordingly to the physical condition of the patient and his/her activity. Such regulation can be for example automatically or manually, but also it can occur continuously or discontinuously, e.g., in intervals. For example, a minimal pressure can be specified of e.g., 2 mmHg, 4 mmHg, 6 mmHg etc. or for example continuously, e.g., by a pressure cuff, a pressure hull, a regulable spring element etc., depending on the age, the condition of the patient, the measurement area and position of the patient (e.g., during an operation or physical activity) the sensor pressure specified and regulated accordingly. It should be noted that e.g., after a physical activity the body has a rest phase lasting for several minutes. During this phase, physiologically reasoned, the venous pressure drops. If during this phase the contact pressure is too high, the blood values will not correspond to the blood values in the rest of the body. Depending on the age and condition of the person this is more or less distinct. Depending on the physical activity or rest phase after sports, sleep, etc. the sensor pressure must be increased or lowered during this time, so that the sensor has a correct contact with the skin and concurrently, the veins are not crushed or clamped. For this the sensor pressure must be right. From a medical point of view, it can be explained as follows:

The venous pressure changes because the small and bigger muscle fibers next to the veins support the blood flow, and consequently the further transport of blood. Further the faster pulse and the higher blood pressure reasoned by physical activity increase during this time the venous pressure and support the further transport of blood as well. Thus, the sensor pressure should be increased during a sports activity, in young healthy patients at e.g., up to 30 mmHg. Conversely, the sensor pressure must be lowered after a physical activity or during a rest phase. If the contact pressure was always only e.g., 2 mmHg, the sensor would constantly be taking off from the skin surface while jogging. Then, a correct measurement would also not be possible.

An apparatus for regulation of the spring force of a spring is known e.g., from EP0651173. For example, also more spring elements can be connected in parallel, so that a bigger spring constant results in total, or a parallel connection can be abrogated to achieve a smaller spring constant. Also, a series connection (e.g., hanging on several springs to each other), or an abrogation of such a series connection, is conceivable. Thereby the spring combination has a smaller spring constant (it is softer) than the softest single spring. Also, the spring can be adjusted by e.g., providing the measurement device as a set comprising at least two spring elements suitable for, or designed for mounting the at least one sensor. In case that the at least two spring elements have a different spring constant, a regulation of the contact pressure can occur by a simple exchange of the one to the other spring element. Both at the same and different spring constant of the at least two spring elements a regulation can be made by a series or parallel connection. Such regulations can be made mechanically and also automatically.

In an embodiment of the measurement device which can be combined with every already mentioned embodiment and every embodiment subsequently mentioned, unless in contradiction, the measurement device is characterized in that the measurement device, in particular the means for pressure regulation, comprise at least one further sensor for the determination of the external force and/or the contact pressure, for example a force sensor, a pressure sensor, a movement sensor, or acceleration sensor, or a position sensor. In this way it is possible to regulate the means for pressure regulation depending on the measured external force and/or the contact pressure.

In an embodiment of the measurement device which can be combined with every already mentioned embodiment and every embodiment subsequently mentioned, unless in contradiction, the measurement device is characterized in that the measurement device, in particular the means for pressure regulation, comprise at least one of the following to regulate the level of damping, or the elasticity of the mounting:
Servomotor,
Torsion bar,
Camshaft,
Spindle,
Eccentric disc,
Solenoid valve, or solenoid (coil) and ram,
One or more magnets or electromagnets (movable against each other),
Electrode, or electrode pair, in particular (plate) capacitor,
Tension spring,
Pressure spring,
Membrane,
Hydraulic or pneumatic element, such as for example a piston, a cylinder, hyperbaric chamber or air cushion, a gas strut,
Mechanical component made of an electrically deformable material (that is electroactive material of which the hardness and/or form is modifiable by applying electrical voltages), in particular made of an electroactive elastomer or piezo material (generally a so-called smart/intelligent material).

In an embodiment of the measurement device which can be combined with every already mentioned embodiment and every embodiment subsequently mentioned, unless in contradiction, the measurement device is characterized in that the at least one sensor comprises at least one pressure measuring unit. This pressure measurement unit can be applied also e.g. (along with the measurement of one or more vital parameters) for the measurement of the contact pressure of the sensor on the skin surface.

The pressure measurement unit is e.g., designed in form of strain gauges. Such strain gauges are available in rectangular but also round variants, e.g., with a diameter of 8 mm. The pressure measurement unit can be arranged on, or at the sensor in contact with the body, but it can also be installed in a pneumatic pressure unit. Alternatively, the pressure measurement unit can also be mounted, or arranged under, or over the spring element. If the sensor is equipped with a pressure measurement unit, e.g., the minimal and maximal contact pressure, or sensor pressure can be determined. This pressure measurement unit can be activated for example by skin contact. If the contact pressure drops below a predefined minimal contact pressure, e.g., 2 mmHg, no measurement will be performed or the measurement result specially marked.

In an embodiment of the measurement device which can be combined with every already mentioned embodiment and every embodiment subsequently mentioned, unless in contradiction, the measurement device is characterized in that the at least one sensor is mounted by at least one spring element in a recess of the base body, in particular by one, two, three, four or more springs, for example metallic helical springs.

To achieve that the at least one sensor is not rigidly connected with the base body, but instead elastically mounted in the base body, or elastically arranged at the base body, a resilient mounting by at least one spring element in a recess of the base body can be expedient. Such a spring element can for example be arranged in the recess and behind, or under the sensor and connect the sensor with the main body. The recess can also be lined with a spring element, for example in the form of an elastomer, a spring, a controlled pressure unit, etc., in which the sensor is embedded. Alternatively, or also additionally, the sensor can be tensioned between at least two springs which are each connected on a side with the base body over the recess and each on a side with the sensor. Of course, also 2, 3, 4, 5, 6, 7, 8, etc. spring elements can be applied. In particular, a fastening over four diagonally tensioned spring elements in a recess with rectangular base form results in an especially stable and uniform mounting of the sensor. An addition or removal of a spring element influences the level of elasticity with which the sensor is mounted or arranged. As already discussed, the term “spring element” is to be understood broadly. It can be a body (e.g. ball, rectangle) made of an elastic material, such as e.g. an elastomer, but also classical helical springs or membranes of metal or another suitable material are conceivable. Moreover, among others also pneumatically or mechanically controlled pressure or spring elements are included.

In an embodiment of the measurement device which can be combined with every already mentioned embodiment and every embodiment subsequently mentioned, unless in contradiction, the measurement device is characterized in that the at least one spring element is an elastic band (or a membrane).

Instead of or together with classical helical springs in particular elastic bands can be used for the resilient mounting of the sensor. In particular, the wearing comfort can be increased if elastic bands, such as for example gums, will be in contact with the skin of the patient instead of metallic helical springs. For example, the spring strength can be adjusted by using more or less elastic bands for the mounting of the sensor, or else, different elastic bands are applied (e.g. broader or thicker and/or made of a more or less elastic material). The color of the bands can be used e.g. for their differentiation or classification.

In an embodiment of the measurement device which can be combined with every already mentioned embodiment and every embodiment subsequently mentioned, unless in contradiction, the measurement device is characterized in that the at least one sensor is plane, bent and/or flexible.

Depending on the position and person, the sensor can have a different geometry to correctly provide for the contact pressure. It is advantageous, when in particular large-area sensors have a slight bending and are not completely planar to better adapt to the anatomy of the patients' body part on which it should be measured. Depending on specified measurement area the bending can be more or less distinct. Especially sensors with a small area, or which are intended to measure e.g. on the thigh or in the chest area, and not on the finger, wrist or upper arm can be flat-shaped without any disadvantage. Particularly practicable are flexibly designed sensors because they are universally applicable and thus provide a correct contact pressure irrespectively of the measurement area on the body of the patient.

An aspect of the invention pertains to a measurement system according to claim 13.

Such a measurement system comprises a measurement device according to the invention and a computer connected with the measurement device. Said computer is either arranged at the measurement device or designed separately, in particular as a mobile phone, and suitable for input of patient-related information and/or reading of measured vital parameters. The patient-related information comprises in particular age, sex, fitness condition, pre-existing conditions, such as for example diabetes or COPD, or skin texture. The connection with the measurement device applies in particular to the at least one sensor for data reading, and/or the spring element resp. the mechanism/the apparatus for the adjustment of the spring constant and herewith of the contact pressure, and/or, if available, the pressure measurement area of the sensor or a separately designed measurement pressure sensor, and/or, if available, the at least one movement or acceleration sensor, and/or, if available, the at least one position sensor.

If the measurement device is used in the orbit or the space, it is a good idea to enter also the gravity using the computer. And in case that the patient is not human but an animal, also the animal race should be input.

It is the computer which then adjusts the contact pressure of the sensor by the connection with the measurement device and based on the patient-related information. In addition to the already mentioned patient-related data the contact pressure can be adjusted even more precisely by the computer when the latter also includes in its calculations the data such as the physical activity (e.g. sport or sleep) or even the wearing position of the sensor on the body (e.g. upper arm, wrist etc.). Data on the physical activity can be for example electronically or mechanically collected, by the measurement device comprising for example an additional sensor being a movement sensor which is also connected with the computer. The wearing area can be input analogous to the rest of patient-related data. Instead of or additionally to, or in combination with a movement sensor, the measurement device can also comprise a position sensor. The latter can recognize if the measurement device is in horizontal or vertical position and allow conclusions to be drawn as to whether the carrier is e.g. lying or standing. This information can again be used to regulate the contact pressure. For example, a decrease can occur as soon as a horizontal position was recognized for a certain amount of time, such as for example 10 min, and the contact pressure so switched to “relax mode”. Since position and movement sensor do not serve the purpose of determining a vital parameter, they do not have to or are preferably not springy mounted.

Depending on the physical condition of a person and its activity, the contact pressure and, if necessary, also the measurement area must be adjusted or preset. Also, a calibration of the measurement unit with a reference device can be made to compensate deviations e.g. from the skin color, skin thickness, age, pre-existing condition, skin tension etc., because these parameters are patient-related and can have an influence on the result of the measurement. These parameters can be pre-programmed as reference data or basic data in the computer.

With the data measured by the sensor and the information provided by the manual input, the necessary contact pressure for the sensor is calculated and adjusted. The reason therefor is that the maximal venous pressure or the pressure which can be applied on the skin surface by the sensor until the blood supply under the sensor does not anymore correspond to the rest of the blood composition, is not constant. The maximal venous pressure varies depending on condition of the body (e.g. sleep, sport, rest phase after sport etc.) and depending on the physical condition of the patient, such as: age, skin texture (skin damaged e.g by sun) etc. The contact pressure of the sensor must be adjusted accordingly to this maximal venous pressure. The correct contact pressure on the skin is crucial for the values to be measured such as: blood sugar, O₂, blood pressure, cardiological conditions etc. The measured vital parameters which can be extracted from the computer, or which can be read by the computer are e.g. real-time data, wherein the measurement can occur also 24/7. In such a case the measurement system can have e.g. an alarm function and/or an automatic SOS function which triggers in case of symptoms or events such as e.g. a stroke, hypoglycemia, a physiological collapse, strong drop in blood pressure, as e.g. in the event of an accident. In this way either the awareness of the direct environment can be raised, or help can even be requested immediately.

A further aspect of the invention pertains to a method for the pressure determination, with which the sensor of a measurement device according to an invented measurement system lies on the skin of the patient, according to claim 14. In said method the pressure, the so-called contact pressure, is calculated with the help of the computer of the measurement system. An algorithm, suitable for determining the optimal contact pressure based on at least one patient-related information, is saved in the computer. This algorithm can determine the optimal contact pressure for example iteratively, e.g. by executing an algorithm multiple times to gradually approach the optimum contact pressure. Optionally, in addition to the at least one patient-related information also at least one further value can be processed by the algorithm. Eligible are e.g. movement or acceleration, time, position of the sensor relative to the standard fixation position at the body and gravity. For example, the measurement device can have a basic setting which is adjusted to a young, sporty patient with a BMI of 20 and who is a non-smoker. In case of sporting activity, a contact pressure of 30 mmHg, at normal activity of 25 mmHg, while sitting of 20 mmHg and while sleeping or in the rest phase after the sporting activity of 18 mmHg, would be preset. For a middle-aged patient who is a non-smoker and sporty but, with a BMI of 25, slightly overweight, e.g. the following contact pressures would be preset: while sporting activity 28 mmHg, at normal activity 23 mmHg, while sitting 18 mmHg and while sleeping or in the rest phase after the sporting activity 16 mmHg. For an older age patient who is a non-smoker and sporty but, with a BMI of 25, also slightly overweight, e.g. the following contact pressures would be preset: while sporting activity 23 mmHg, at normal activity 20 mmHg, while sitting 16 mmHg and while sleeping or in the rest phase after the sporting activity 11 mmHg. For an older age patient who is a smoker and has a BMI over 30 e.g. the following contact pressures would be preset: while sporting activity 20 mmHg, at normal activity 16 mmHg, while sitting 11 mmHg and while sleeping or in the rest phase after the sporting activity 6 mmHg. In case that a comparable patient is also under medical treatment, e.g. the following contact pressures would be preset: while sporting activity 18 mmHg, at normal activity 14 mmHg, while sitting 10 mmHg and while sleeping or in the rest phase after the sporting activity 4 mmHg.

In general, the more measurement values and patient-related information are included in the determination of the contact pressure, the more precisely the latter can be determined.

To check whether the contact pressure was correctly chosen and also for calibration and further adjustment of the contact pressure, a sensor lifting and anew lowering can be done. If the contact pressure is adjusted correctly the sensor will measure the same value (including tolerances) before the lifting and after the anew lowering or pressing, and so the contact pressure is adjusted correctly. In case that a change of the measurement value, in particular an increase, are observed, the contact pressure seems to be set too high and the blood flow thereby limited. Accordingly, the contact pressure must be decreased. This can be done manually, but also automatically by a control circuit. Analogously, such a control circuit can also be used to optimize the contact pressures specified by the algorithm or to calibrate. In case that the specified contact pressure is found to be in reality too high, the algorithm can be adjusted by the control circuit so that it will output a smaller contact pressure for the same conditions in future. Of course, alternatively to the automatic control circuit, also a manual calibration can be done here.

The algorithm can e.g. stipulate for inspection that in the first week of wearing the measurement device while a sporting activity, every 30 min the sensor is lifted and again lowered (and hence again placed with the previous contact pressure), while a normal activity, the sitting and the sleep resp. the rest phases after sport this takes place every 60 min. At a difference of e.g. 0.2% to 0.5% (e.g. 0.2%, 0.3%, 0.4%, 0.5%) of the oxygen saturation (SPO₂) the contact pressure will be decreased by e.g. 2 mmHg, in particular for the activity during which the difference has been determined. In the second week of wearing the measurement device the sensor is lifted during a sporting activity every 45 min and again lowered, while a normal activity every 120 min, while sitting and sleeping resp. the rest phases after sport every 90 min. Also here, at a difference of e.g. 0.2% to 0.5% of the oxygen saturation (SPO₂) the contact pressure will be decreased by e.g. 2 mmHg, in particular for the activity during which the difference has been determined. Provided that no important adjustment had to be done in the second week, in the third week of wearing the measurement device the sensor can be lifted during a sporting activity e.g. every 30 min/60 min/2 h/4 h/6 h/24 h and again lowered. The same applies while a normal activity, while sitting and sleeping resp. the rest phases after sport. Also here, at a difference of e.g. 0.2% to 0.5% of the oxygen saturation (SPO₂) the contact pressure will be decreased by e.g. 2 mmHg, in particular for the activity during which the difference has been determined.

A further aspect of the invention pertains to a method for the measurement of at least one vital parameter according to claim 15. Said method comprises the step of arranging a measuring device according to an invented measurement system on a patient and either entering of at least one patient-related information in the computer of the measurement system or retrieving of at least one saved patient-related information from the computer of the measurement system. Further the method comprises the step of adjusting the pressure with which the sensor of the measurement device lies on the skin, the so-called contact pressure, based on the at least one patient-related information.

Optionally, alongside the at least one patient-related information also at least one further value can be entered, read, or measured to then also be included in the adjustment of the pressure. Eligible values are e.g. movement or acceleration, time, position of the sensor relative to the standard fixation at the body and gravity.

In an embodiment of the method which can be combined with every already mentioned embodiment and every embodiment subsequently mentioned, unless in contradiction, the method further comprises carrying out an initial measurement to obtain at least one initial measurement value to adjust the pressure with which the sensor of the measurement device lies on the skin, based on the at least one patient-related information and the at least one initial measurement value.

Also here, optionally alongside the at least one patient-related information also at least one further value can be entered, read, or measured to then also be included in the adjustment of the pressure.

A further aspect of the invention pertains to the use of a measurement device according to the invention, according to claim 20. Such a use of the measurement device is aimed at the determination of at least one vital parameter. E.g. blood pressure, pulse, blood sugar, oxygen saturation, temperature, humidity and skin texture count to the vital parameters.

In an embodiment of the use which can be combined with every already mentioned embodiment and every embodiment subsequently mentioned, unless in contradiction, the use further aims at the determination of at least one further value. The at least one value is among others the movement or acceleration of the patient, the pressure of the sensor on the skin alias contact pressure, the time (in particular measurement time and/or measurement duration and/or time), the position of the sensor relative to the standard fixation at the body (e.g. standing, lying) and the gravity.

Embodiments of the present invention are explained in more detail based on the following figures.

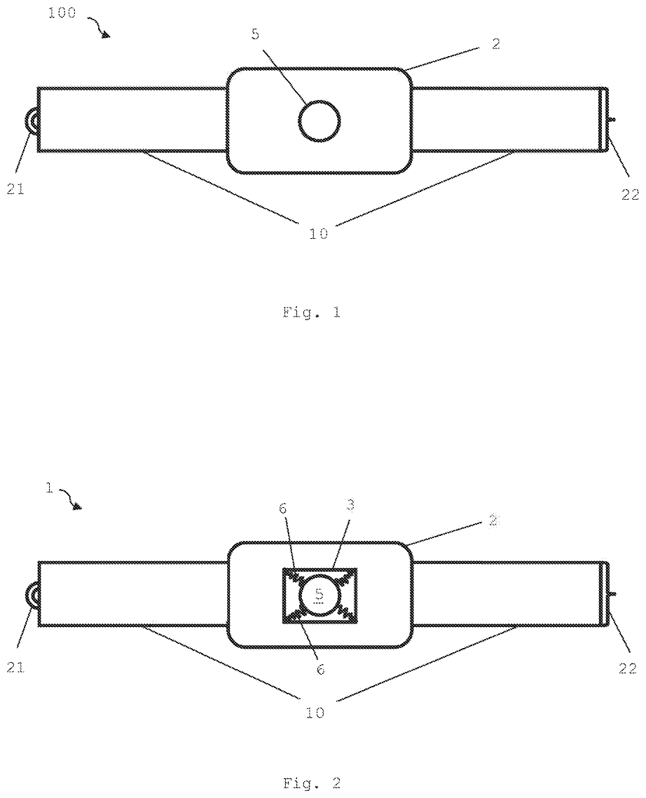

FIG. 1 a schematic depiction of a measurement device for vital parameters according to the state of the art;

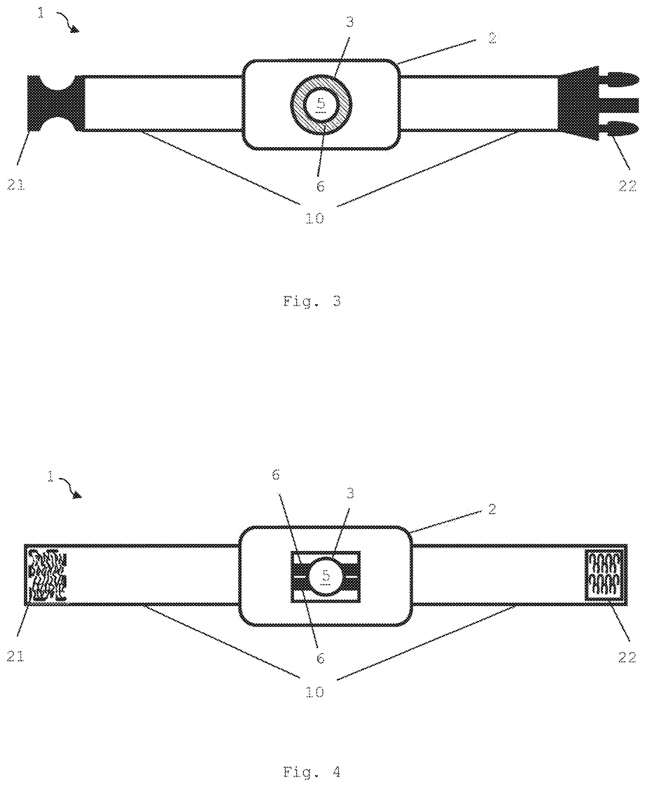

FIG. 2 a schematic depiction of a measurement device for vital parameters according to the invention;

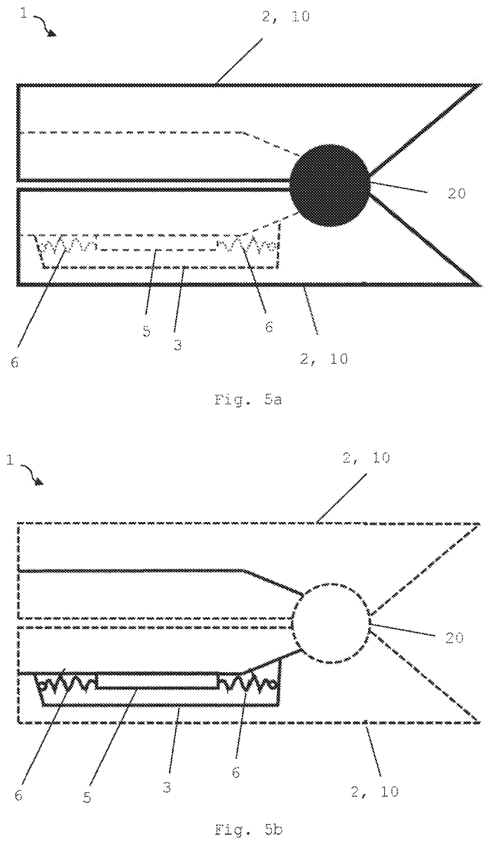

FIG. 3 a schematic depiction of an embodiment of a measurement device for vital parameters according to the invention;

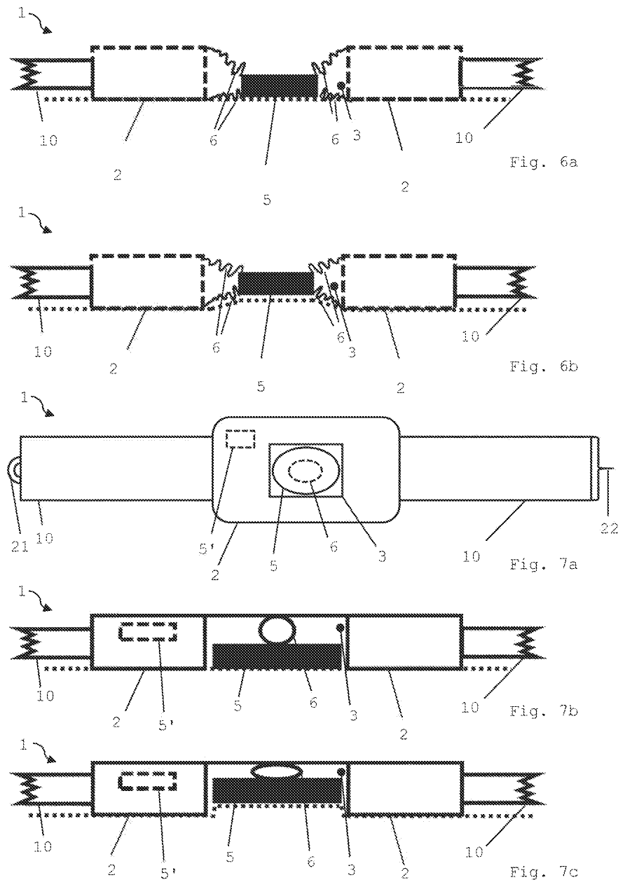

FIG. 4 a schematic depiction of an embodiment of a measurement device for vital parameters according to the invention;

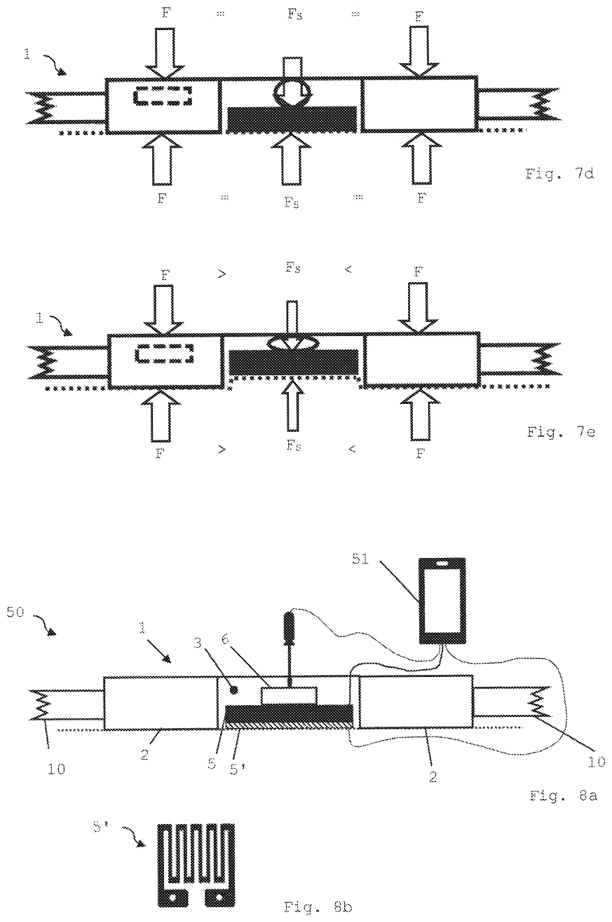

FIG. 5a a schematic side view of an embodiment of a measurement device for vital parameters according to the invention;

FIG. 5b a schematic longitudinal section of the embodiment of a measurement device for vital parameters shown in FIG. 5a;

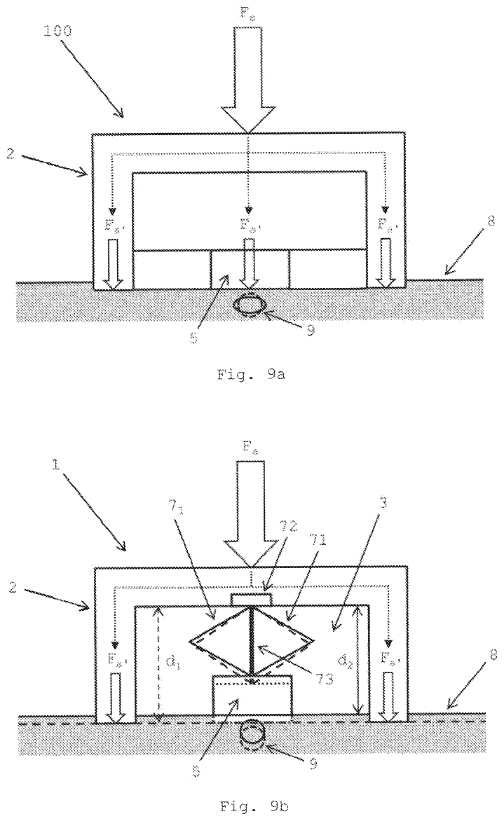

FIG. 6a a schematic longitudinal section of a measurement device for vital parameters according to the invention in a first position of the sensor;

FIG. 6b a schematic longitudinal section of a measurement device for vital parameters according to the invention in a second position of the sensor;

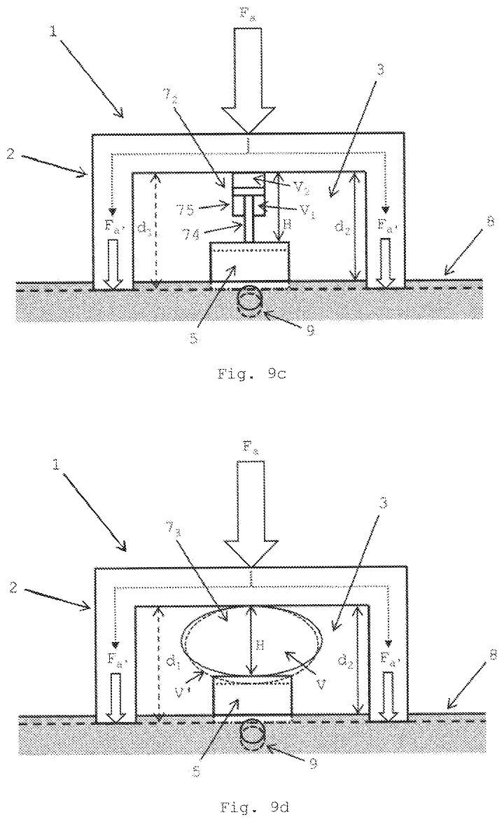

FIG. 7a a schematic depiction of an embodiment of a measurement device for vital parameters according to the invention;

FIG. 7b a schematic longitudinal section of the embodiment of a measurement device for vital parameters according to the invention shown in FIG. 7a in a first position of the sensor;

FIG. 7c a schematic longitudinal section of the embodiment of a measurement device for vital parameters according to the invention shown in FIG. 7a in a second position of the sensor;

FIG. 7d a schematic longitudinal section analogous to FIG. 7b with forces drawn in;

FIG. 7e a schematic longitudinal section analogous to FIG. 7c with forces drawn in;

FIG. 8a a schematic depiction of a measurement system for vital parameters according to the invention;

FIG. 8b a schematic depiction of a pressure measurement sensor;

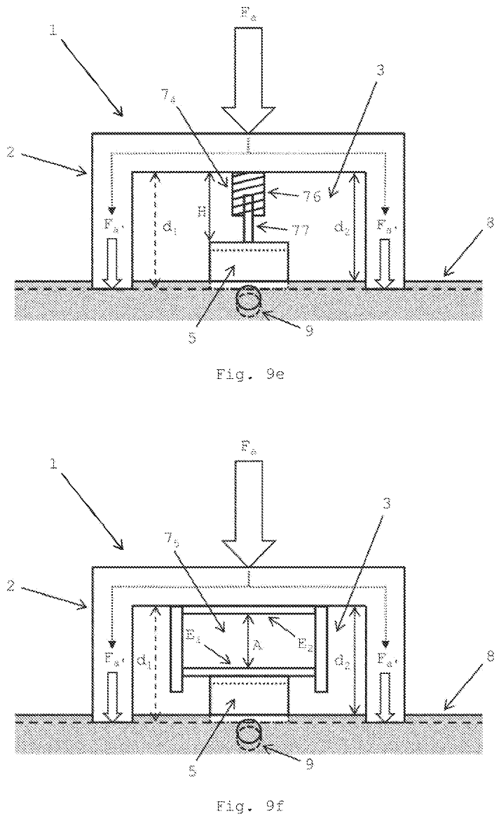

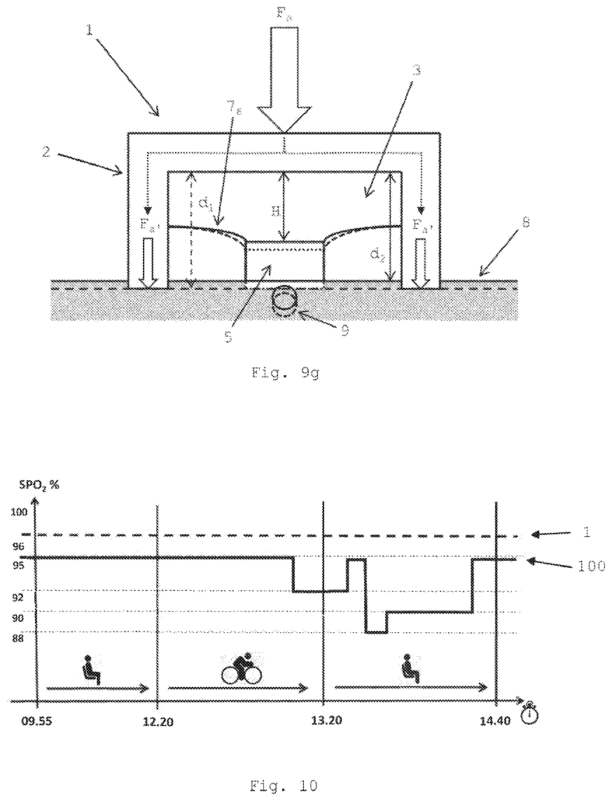

FIG. 9a a schematic longitudinal section of a measurement device for vital parameters according to the state of the art in which the sensor is rigidly connected with the case (or the base body);

FIG. 9b a schematic longitudinal section of an exemplary measurement device for vital parameters according to the invention with a servomotor-based means for pressure regulation;

FIG. 9c a schematic longitudinal section of an exemplary measurement device for vital parameters according to the invention with a piston-based means for pressure regulation;

FIG. 9d a schematic longitudinal section of an exemplary measurement device for vital parameters according to the invention with an air cushion-based means for pressure regulation;

FIG. 9e a schematic longitudinal section of an exemplary measurement device for vital parameters according to the invention with a solenoid valve-based means for pressure regulation;

FIG. 9f a schematic longitudinal section of an exemplary measurement device for vital parameters according to the invention with a capacitor-based means for pressure regulation;

FIG. 9g a schematic longitudinal section of an exemplary measurement device for vital parameters according to the invention with a membrane-based means for pressure regulation; and

FIG. 10 a schematic depiction of a comparative measurement between a measurement device 100 from the state of the art and a measurement device 1 according to the invention.

FIG. 1 shows a measurement device for vital parameters 100 known from the state of the art, shown in a view that allows the view on the site of the measurement device 100 which for the measurement is laid onto the skin of the patient, the so-called underside. The measurement device 100 comprises a base body 2 in which the at least one sensor 5 is embedded for measuring at least one vital parameter and rigidly connected. For example, the sensor 5 can be screwed with the base body 2 or glued in a recess in the base body 2 adapted to the form of the sensor. To at least temporary attach the measurement device 100 to the arm or any another body part of the patient, such as for example of the trunk, the measurement device 100 further comprises a fixing means 10, such as here for example an elastic two-piece band, which can be closed to a ring by a two-piece fastening 21,22. The fastening depicted here corresponds to an eyelet as first fastening piece 21 and a hook as second fastening piece 22. The total length of the measurement device 100 including base body 2, band 10 and the fastening pieces 21,22 is chosen in such a way that it sits tightly or fixed at the specified body part in closed state, so that it cannot slip, optimally also not while moving, as e.g. a physical activity such as jogging or the like. Alternatively, the measurement device 100 can also have a means for the adjustment of the total length of the measurement device 100. For example, at least one or both pieces of the band 10 are adjustable in length (note: it is not the case in the shown measurement device 100). The fixed sit of the measurement device 100 at a body part of the patient ensures that not only the band 10 and the base body 2 of the measurement device 100 are tightly pressed onto the skin of the corresponding body part, but that also the sensor 5 presses on it with the same force.

Consequently, the resulting interaction between the measurement device and the system to be measured ensures that an intervention in the system takes place changing it, so that measurement device 100 determines a value for a vital parameter which maybe would be correct as single value, but which does not correspond to the measurement value present in the rest of the body, and which is intended to be measured. The actual value is after all the one that is present in the body when no interaction takes place.

FIG. 2 shows a measurement device for vital parameters 1 according to the invention. The latter is also shown in a view that allows the view on the site of the measurement device 1 which for the measurement is laid onto the skin of the patient, the so-called underside. The measurement device 1 comprises a base body 2 at which, on two opposite sides, an elastic two-piece band 10 is arranged for the fixation at a body part. This two-piece band 10 can be for example closed by a hook-and-eyelet closure 21,22. On the contrary to the previously known measurement device shown in FIG. 1, the sensor 5 of the measurement device according to the invention of FIG. 2 is not rigidly connected with the base body 2. Moreover, the sensor 5 is elastically mounted in a recess 3 in the base body 2 by four spring elements 6 (for the sake of clarity only two of the four springs are marked by reference signs). In the shown embodiment the recess 3 has a rectangular ground view and the springs 6 are tensioned starting from the edges of the recess 3 up to the here round sensor 3. In an alternative embodiment the sensor 5 can be mounted with also e.g. 1, 2, 3, 4, 5, 6 etc. spring elements 6. The spring elements 6 illustrate here a movable and elastic element, which could also be an elastomer etc.

FIG. 3 shows an embodiment of a measurement device for vital parameters 1 according to the invention. The latter is also shown in a view that allows the view on the site of the measurement device 1 which for the measurement is laid onto the skin of the patient, the so-called underside. The measurement device 1 comprises a base body 2 at which, on two opposite sides, an elastic two-piece band 10 is arranged for the fixation at a body part. This two-piece band 10 can be for example closed by a snap-fit 21,22. Also here the sensor 5 is not rigidly connected with the base body 2. Moreover, the sensor 5 is elastically mounted by a spring element 6. In the shown embodiment the recess 3 has a round ground view in which the spring element 6, here a mass of an elastomer, is recessed, in which mass the sensor 5 is embedded.

FIG. 4 shows an embodiment of a measurement device for vital parameters 1 according to the invention. The latter is also shown in a view that allows the view on the site of the measurement device 1 which for the measurement is laid onto the skin of the patient, the so-called underside. The measurement device 1 comprises a base body 2 at which, on two opposite sides, an elastic two-piece band 10 is arranged for the fixation at a body part. This two-piece band 10 can be for example be closed by a velcro 21,22. Also here the sensor 5 is not rigidly connected with the base body 2. Moreover, the sensor 5 is elastically mounted in a recess 3 in the base body 2 by two spring elements 6 in form of two elastic bands. These are terminally connected with the recess 3 and centrally connected with the sensor 5, behind which they pass through. Instead of a band 6 that passes behind the sensor 5 also two elastic bands 6 can be applied, which then are arranged each via one site at the base body 2 and each via the other site at the sensor 5. In this way the elastic band 6 does not have to be passed behind the sensor 5.

FIG. 5a shows an embodiment of a measurement device for vital parameters 1 according to the invention in a side view. This measurement device 1 is especially suitable for the measurement on the finger of a patient. Base body 2 and fixing means 10 are here one and are formed by the upper site and the underside of a clamp which can be opened and closed as closure by a spring 20. The elements which actually would not be visible in the side view are dashed. Hereto count, next to the material recess for the insertion of the finger (without reference sign), the recess 3 in which the sensor 5 is elastically mounted by e.g. two spring elements 6.

FIG. 5b shows the embodiment of a measurement device for vital parameters 1 according to the invention, shown in FIG. 5a, in a longitudinal view. Especially easy to see are in this depiction the recess 3 in which the sensor 5 is elastically mounted by two spring elements 6. Also, the anchoring of the spring elements 6 in the recess 3 of the bottom part of the clamp alias base body 2 and fixing means 10, schematically depicted as two eyelets, are clearly visible.

FIG. 6a shows a measurement device 1 according to the invention in a first position of the sensor 5 in a longitudinal view. FIG. 6b however shows the same measurement device 1, also in longitudinal view, but the sensor 5 is here in a second position. The measurement device 1 is comparable to the one in FIG. 2 with the difference that the sensor 5 is elastically mounted in the recess 3 of the base body 2 not only by four but by eight spring elements 6, wherein in the longitudinal view only four of these spring elements 6 are visible. The spring elements 6 are arranged in a manner that four of the spring elements 6 generate a pull up (so from the underside, that is away from the site of the measurement device 1 which contacts the skin while using) and four of the spring elements 6 generate a pull down (so to the underside). The base body 2 at which, on two opposite sides, an elastic two-piece band 10 is arranged for the fixation at a body part, appears in the longitudinal view as two pieces because of the recess 3, but in reality, it is formed as one piece. Unlike in FIG. 2, the elastic two-piece band 10 is not depicted in full length, as illustrated by the frayed ends. Accordingly, the closure is not visible. In said first position the spring elements 6 (here in the picture at the bottom), which are facing the skin of the patient (skin surface is schematically depicted as dotted line) are more tensioned than in the second position shown in FIG. 6b. Consequently, while wearing the measurement device 1 in the first position a stronger contact pressure is applied onto the skin of the patient than in the second position. In other words, the measurement device 1 with a sensor 5 mounted in the first position (sensor 5 at the same height as the base body 2 referenced to the site facing the skin of the patient) is more suitable for e.g. younger patients or while physical activities, that is in the case of a higher venous pressure. The measurement device 1 with the sensor 5 in the second position (sensor 5 pressed inwards resp. in the drawing pressed up, so with distance to the base body 2 and not flush with the base body 2 referenced to the site facing the skin of the patient) is however better suitable for e.g. older or resting patients with a lower venous pressure. The reason therefor is that the skin can expand in the generated recess (sensor 5 is pressed inwards) and thus the pressure under the sensor decreases.

FIG. 7a shows an embodiment of a measurement device for vital parameters 1 according to the invention. The latter is shown in a view that allows the view on the site of the measurement device 1 which for the measurement is laid onto the skin of the patient, the so-called underside. The measurement device 1 comprises a base body 2 at which, on two opposite sides, an elastic two-piece band 10 is arranged for the fixation at a body part. This two-piece band 10 can be for example closed by a hook-and-eyelet closure 21,22. The sensor 5 is elastically mounted in a recess 3 in the base body 2 by a spring element 6. The spring element 6 is dashed because—viewed from the bottom—it is behind the sensor 5 and connects there the sensor 5 with the base body 2 via the recess 3. Also dashed is a second sensor 5′ which is e.g. a movement sensor and is not elastically mounted but rigidly embedded in the base body 2.

FIG. 7b shows a longitudinal view of the measurement device 1 depicted in FIG. 7a in a first position of the sensor 5. FIG. 7c however shows a comparable measurement device 1, also in longitudinal view, but the sensor 5 is here in a second position. The base body 2 does not seem like it is divided into two parts also in the longitudinal view despite the recess 3 because the recess 3 of this measurement device 1 is not a continuous opening in form of a clearance hole, but it only represents kind of a notch in the bottom part of the base body 2, in which notch the spring element 6 and then the sensor 5 were introduced. However, the second sensor 5′ is directly framed in the base body 2 and it is not mounted in the recess 3. The elastic and two-pieced band 10 is only partially depicted, comparable to the FIGS. 6a and 6b. In said first position (see FIG. 7b) the spring element 6 is a strongly resp. stronger adjusted means for pressure regulation, the sensor 5 reaches up to the edge of the bottom part respectively the skin surface (dashed line). If the measurement device 1 is applied to the patient, the base body 2 presses with a first pressure onto the skin surface. However, the sensor 5, which is elastically mounted, only presses with a second, lower pressure onto the skin surface because thanks to the spring element 6 (e.g. a spherical elastomer) not the full fixing force of the base body 2 is transferred to the sensor 5 and thus pressure is taken away from the skin area where the at least one vital parameter should be measured. The choice of the spring element 6 can be used to set the contact pressure with which the sensor 5 finally lies on the skin.

The round spring 6, as depicted in FIG. 7b, corresponds to a strongly resp. strong adjusted means for pressure regulation (7) so that the measurement device 1 is suitable for higher venous pressures, and the oval spring 6, as depicted in FIG. 7c, corresponds to a weakly resp. weaker adjusted means for pressure regulation (7) so that the measurement device 1 in this case is suitable for lower venous pressures.

The FIGS. 7d and 7e show the measurement device 1 with sensor in a first and a second position analogous to the FIGS. 7d and 7e but the present force relationships, which define the contact pressure, are marked, the rest of the reference signs was not depicted for the sake of clarity. Marked are force F (force from the top—arrow points downwards) and counterforce F (force from the bottom—arrow points upwards), which are defined by applying the measurement device 1 to the patient and the strength of its fixation. Additionally, force and counterforce Fₛ which are present in the area of the sensor are marked. With a relaxed spring element (FIG. 7d) the forces and counterforces Fₛ are evenly distributed across the whole measurement device 1. So the same pressure prevails in the area of the sensor, as it is predefined by applying the measurement device 1. It is visible in FIG. 7e that the force and counterforce Fₛ, and thus also the contact pressure is lower in the area of the sensor than the force and counterforce F in the area of the rest of the base body 2. By pressing together the spring element, the latter absorbs a part of the force F predefined by applying the measurement device 1 and so enables a lower contact pressure below the sensor.

FIG. 8a shows a measurement system for vital parameters 50 according to the invention. The shown embodiment of the measurement device 1 according to the invention of this measurement system 50 comprises a base body 2, which by fixing means 10 (only partially depicted, as illustrated by the jagged terminal lines) is attachable to the body of a patient. The base body 2 comprises a recess 3 in which the sensor 5 is elastically mounted by a spring element 6. The spring constant of the spring element 6 is adjustable, as symbolically implied by the screwdriver. Below the sensor 5, so between the sensor 5 and the skin surface of the patient (dashed line), another sensor 5′ is arranged. The latter is a pressure measurement sensor 5′ which can determine the contact pressure of the sensor 5. The pressure measurement sensor 5′ can extend along the whole surface facing the skin surface of the patient or else only along a part of this surface (note: optical measurements through a measurement pressure sensor 5′ as depicted in FIG. 8b are possible without any problems). The measurement pressure sensor 5′ can also be formed as a pressure measurement unit comprised in the sensor 5. Connected to the measurement pressure sensor 5′ and the spring element 6, or the apparatus for adjusting the spring constant of the spring element and also the sensor 5 (here depicted by physical connections—of course any kind of wireless communication is also possible) is a computer 51, here exemplary represented by a mobile phone 51. The latter is capable of setting the contact pressure of the sensor 5 by the spring element 6 based on the patient-related information deposited in the mobile phone 51 and the provided algorithm. Moreover, the mobile phone 51 can read or save and/or depict the at least one measured vital parameter via the connection to the sensor 5. Because of the measurement pressure sensor 5′, which is optional in the shown embodiment, and the connection to this measurement pressure sensor 5′, the mobile phone 51 can also monitor the actual prevailing contact pressure and if necessary, correctively intervene. If the predetermined contact pressure and the actual prevailing contact pressure are different, the spring constant of the spring element 6 can be adjusted accordingly, so that actual value and nominal value do match.

FIG. 8b shows an embodiment of a measurement pressure sensor 5′ as it can e.g. be applied in a measurement device according to the invention.

FIG. 9a shows a schematical longitudinal view of a measurement device 100 for vital parameters as known from the state of the art, in which the sensor 5 is rigidly connected with the casing (or the base body 2). The fixing means 10 are not marked in the FIG. 9a. In measurement devices 100 according to the state of the art the sensor 5 protrudes from the base body 2 and is, as visible in FIG. 9a, flush with the underside of the base body 2, which is laid on the skin. If an external force Fₐ acts on the base body 2, this external force Fₐ will be transferred to the underside of the base body 2 incl. to the sensor 5, where it acts as force F_{a′} on the skin surface. In this way the underside of the base body 2 is pressed together with the within rigidly arranged sensor 5 in the skin 8 lying below, resulting in that the vein 9 is squeezed and the blood flow through the vein 9 is affected or even stopped. Thereby also a measurement of vital values which are dependent from (largely undisturbed) blood flow through the vein 9 will be falsified, which of course is extremely undesirable.

In the following, solutions according to the invention are described, in which the sensor is mounted in an actively damped manner in the base body by the means for pressure regulation, in particular in a recess of the base body. The active damping of the sensor ensures that the contact pressure of the sensor onto the skin in the measurement area is (mostly) adjustable independently from an external force acting on sensor, or it is adjustable depending on the latter. Therefor e.g. the contact pressure is continuously monitored by a sensor and accordingly adjusted by an actuator of the means for pressure regulation. It means that a regulator ensures that the contact pressure remains constant, (mostly) independent from an external force acting on the base body, or it is always equal to a desired (e.g. adjustable) value. Alternatively, several components of the external force in the three spatial directions can be detected and equilibrated or eliminated e.g. by a triaxial (3D) acceleration sensor.

FIG. 9b schematically illustrates a longitudinal view of an exemplary measurement device 1 for vital parameters according to the invention in which the above-mentioned problem of the transfer of an external force Fₐ to the sensor 5 is reduced, or even completely avoided by providing a means for pressure regulation 7 to the measurement device 1 according to the invention with which the sensor 5 is damped mounted in the base body 2, or as shown in FIG. 9b, it is damped arranged in a recess 3 of the base body 2. In the example of FIG. 9b a trapezoidal construction 71 is used as means for pressure regulation 7₁, of which the height is adjustable by a servomotor 72 via a spindle 73, similar to a scissor jack. The contact pressure of the sensor 5 onto the skin 8 is measured by the sensor 5 or by a separate pressure sensor and depending on whether this contact pressure is above or below the desired contact pressure the position of the sensor 5 will be accordingly adjusted within the recess 3, it is the distance of the sensor 5 to the ceiling of the recess 3 is accordingly decreased or increased. As soon as an external force Fₐ acts on the base body 2 it is automatically registered and the means for pressure regulation 7₁ ensures that the contact pressure always corresponds to a desired (predetermined or adjustable) value. As visible in the FIG. 9b, the base body 2 is pressed, because of the external force Fₐ, with the force F_{a′} on the edge of the base body 2 on the skin, so that the edge of the base body 2 protrudes into the skin. In order that the external force Fₐ is not transferred to the sensor 5, the latter must be pulled in the recess 3 by the height h=d₁−d₂ by the means for pressure regulation 7₁. As soon as the external force Fₐ is no longer present the sensor 5 is again driven down by h by the means for pressure regulation 7₁. In this way the contact pressure of the sensor 5 onto the skin 8 is continuously monitored and always kept at the desired nominal value with the help of the means for pressure regulation 7₁.

At this point it should be noticed that when multiple sensors 5 in the measurement device 1, it is, are arranged in the base body 2 by damped mounting, for every single each sensor 5 a means for pressure regulation 7 and a corresponding pressure sensor for the measurement of the contact pressure can be applied. Depending on the arrangement and purpose of the individual sensors 5 they alternatively can have a single or partially common means for pressure regulation 7 as well as a single or partially common pressure sensor for measurement of the contact pressure.

It should be further noticed that the electronics which receive and process the measurement signals from the sensor 5 as well as the energy supply for the sensor 5 (e.g. battery, accumulator or “energy harvester”) are detached from it and arranged in the base body 2 in a manner that no force transfer occurs via the necessary connections to the sensor 5. In that way also the mass (and size) of the sensor 5 are especially low so that its weight has only a small influence e.g. on the contact pressure, and the sensor 5 can be moved by the means for pressure regulation 7 without big effort.

Moreover, it should be noted that the damped mounting of the sensor further effects that not only forces vertical to the skin surface but also shear forces are reduced and even eliminated. So, the at least one sensor is mounted in a way that shear forces acting on the base body are transferred only on a reduced extent to no extent to the sensor—and thus to the skin. This is of significant importance because among other factors such as compressive forces shearing strain in the development of a decubitus leads to the shifting of skin layers. The top layer of skin shifts, the lower layers of skin do not shift.

Consequently, this leads to a disorder of the blood circulation and to injuries which are not immediately visible.

FIG. 9c shows a schematic longitudinal view of a further exemplary measurement device 1 for vital parameters according to the invention with an alternatively realized means for pressure regulation 7₂. In the means for pressure regulation 7₂ in FIG. 9c a piston 74 is connected with the sensor 5, wherein the piston 74 is moved in a cylinder 75. If the volumes V₁ and V₂ are changed the piston 74 moves in the cylinder 75 and thus changes the position of the sensor 5 in the recess 3, e.g. the distance H between the sensor 5 and the ceiling of the recess 3. Thus, in turn, the distance H can always be adjusted by a suitable regulator in dependence of the measured contact pressure, so that the contact pressure always corresponds to the desired nominal value. To move the piston for example air or another fluid can be pumped from the one cylinder chamber in the other, so that the two volumes V₁ and V₂ accordingly change in the opposite direction. In addition also (each) a spring could be arranged in the cylinder chamber(s)—similar to the gas spring.

FIG. 9d shows a schematic longitudinal view of a further exemplary measurement device 1 for vital parameters according to the invention with a further alternatively realized means for pressure regulation 7₃. In this case the means for pressure regulation is realized as air cushion. If the volume is reduced from V′ to V, the position of the sensor 5 in the recess 3 changes, that is the distance H between the sensor 5 and the ceiling of the recess 3 decreases because the sensor 5 is connected to the air cushion 7₃. In this way, in turn, the distance H can always be adjusted by a suitable regulator in dependence of the measured contact pressure, so that the contact pressure always corresponds to the desired nominal value. To change the volume V e.g. air (or another fluid) is pumped in or out the air cushion.

FIG. 9e shows a schematic longitudinal view of a further exemplary measurement device 1 for vital parameters according to the invention with a further alternatively realized means for pressure regulation 7₄. In this case the means for pressure regulation is realized as solenoid valve 7₄ composed of an electric coil (solenoid) 76 and a plunger movably arranged in the coil. If the current through the coil 76 is changed, the plunger 77 accordingly moves in or out the latter and changes in this way the position of the sensor 5 in the recess 3, that is the distance H between the sensor 5 and the ceiling of the recess 3 because the sensor 5 is connected to the plunger 77. In this way, in turn, the distance H can always be adjusted by a suitable regulator in dependence of the measured contact pressure, so that the contact pressure always corresponds to the desired nominal value.

FIG. 9f shows a schematic longitudinal view of a further exemplary measurement device 1 for vital parameters according to the invention with a further alternatively realized means for pressure regulation 7₅. In this case the means for pressure regulation is implemented as electrical capacitor 7₃. It is composed of two parallel capacitor plates E₁, E₂ of which the one is connected with the sensor 5 and movable relative to the other capacitor plate arranged on the ceiling of the base body. If electrical charge is shifted by a current from the one capacitor plate E₁ to the other capacitor plate E₂, the electric field between the two capacitor plates E₁, E₂ changes, which then accordingly, more or less attract each other so whereby the capacitor plates E₁ shift relative to capacitor plates E₂ and the distance A between the two capacitor plates E₁, E₂ changes. Accordingly, the distance A can always be adjusted by a suitable regulator in dependence of the measured contact pressure, so that the contact pressure always corresponds to the desired nominal value.

FIG. 9g shows a schematic longitudinal view of a further exemplary measurement device 1 for vital parameters according to the invention with a further alternatively realized means for pressure regulation 7₆. In this case the means for pressure regulation is formed as (spring) membrane 7₆ made of an electroactive material of which the stiffness is modifiable by applying an electric voltage. If e.g. the voltage applied on the membrane 7₆ is increased, the stiffness of the membrane increases thereby lifting the with it connected sensor 5, so that the distance H between the sensor 5 and the ceiling of the recess 3 is reduced. Accordingly, the distance H can always be adjusted by a suitable regulator in dependence of the measured contact pressure, so that the contact pressure always corresponds to the desired nominal value.

At this point it should be noticed that the means for pressure regulation 7 can reduce/compensate not only external forces Fₐ in the vertical direction to the skin surface (as shown in the FIGS. 9b-g) but also that according to the invention, the means for pressure regulation 7 can work in all three dimensions/spatial directions (and thus compensate). In this way it is also possible to (partially) compensate or reduce external forces such as the gravity or forces which act on the sensor due to movements and due to the position of the wearer of the measurement device, and in particular to keep the sensor in a desired measuring position (while sitting, lying—independent from the position or orientation direction of the sensor—and running, where ever-changing (external) movement forces act on the sensor).

FIG. 10 shows a comparison of measurements of oxygen saturation in the blood taken with a measurement device 100 known from the state of the art and a measurement device 1 according to the invention. The male patient on whom the test measurements were taken was 69 years old at the time of measurement and smoker, however not an excessive drinker and no pre-existing conditions such as e.g. COPD or asthma were known. His skin can be described as older and therefor saggy, and damaged from the sun. His fitness level was passable even if not well to very well trained. Nevertheless, his pulse normalizes comparably fast after a physical effort because he was very active in sports in his younger years (skiing, jogging, cycling and body building). The graphic compares the oxygen saturation in [%] which was recorded on one day from 9.55 o'clock to 14.40 o'clock with a conventional measurement device 100 (thick solid line) and a measurement device 1 according to the invention (thick dashed line). The measurement devices were arranged on the upper arm of the patient. During the measurement time, strictly speaking between 12.20 o'clock and 13.20 o'clock, the patient was not resting and instead, he was physically active by cycling. It is clearly visible that the oxygen saturation measured by the conventional measurement device 100 greatly fluctuates (between 88% and 95%), whereas the values measured by the measurement device 1 according to the invention at a contact pressure of ca. 13 mmHg were quasi constant and above 96%. The difference of the values between 95% and 96%, in particular in the rest phase, can happen due to the measurement with two different measurement devices 1, 100 and these have a certain tolerance. However, it also becomes apparent that the conventional measurement device 100 determines in the rest phase an oxygen saturation which tends to be too low. Reason for this would be that the blood flow below the sensor is already affected by the too high contact pressure. The difference becomes seriously and definitively evident in the phases in which the body of the patient comes to rest again after the activity (cool down phase still while sitting on the bicycle and first rest phase while sitting). Although the actual oxygen saturation practically does not change in this phase, the conventional measurement device 100 indicated, misdirected by the changing venous pressure of the patient, a great fluctuation of the oxygen saturation. In particular, in this example the deviation during the physical activity is evident only towards the end because:

A) the contact pressure is relatively low but still above 11 mmHg. The patient is 69 years old.

B) in the beginning different internal venous pressures predominate. The upper arm moves during the exertion (cycling) which supports the blood circulation plus the muscles in the veins. Under physical exertion the pressure of the sensor can be significantly increased because of the movement, the increased pulse, and the blood pressure without any change of the measurement values because the blood flow under the sensor is still guaranteed—also by movement.

C) at the end of the exertion (cycling) there is the cool down phase. The pulse drops from about 145 to 90, then the patient moves, moves his position on the sofa. Finally, when the patient recovered, the values are identical to the initial values, the internal venous pressure stabilized.

D) the adjusted pressure of the sensor compensates these fluctuations in the course of the day.

E) the sensor of the measurement device 100 according to the state of the art has a contact pressure of about 25-30 mmHg (the exact value is difficult to determine because the upper arm has different diameters and the sensor does not always lie the same thereon).

### LIST OF REFERENCE SIGNS

1 measurement device for vital parameters
2 base body
3 recess
5 sensor
6 spring element
7 means for pressure regulation:
   7₁ means for pressure regulation with servomotor and trapezoidal construction (˜scissor jack)
   7₂ means for pressure regulation with piston
   7₃ means for pressure regulation with air cushion
   7₄ means for pressure regulation with magnet valve
   7₅ means for pressure regulation with capacitor (plates)
   7₆ means for pressure regulation with electroactive membrane
71 trapezoidal construction
72 servomotor
73 spindle
74 piston
75 cylinder
76 coil/solenoid
77 plunger
8 skin surface
9 vein
10 fixing means
20 closure
21 first part of the closure
22 second part of the closure
50 measurement system
51 computer
100 measurement device according to the state of the art
A distance of the capacitor plates
d₁ distance between the ceiling of the recess and the skin surface without external force on the base body
d₂ distance between the ceiling of the recess and the skin surface with external force on the base body (<d₁)
E₁ movable capacitor plate
E₂ fixed capacitor plate
F force
Fₛ force sensor
Fₐ external force
F_{a′} external force transferred to the skin surface
H distance of the sensor to the ceiling of the recess
V, V′ volume of the air cushion
V₁ first cylinder volume
V₂ second cylinder volume

1. Measurement device (1) for measuring vital parameters, comprising:
a base body (2);
a fixing means (10); and
at least one sensor (5),
characterized in that the at least one sensor (5) is mounted in a damped manner, in particular actively damped, by a means for pressure regulation (7) in the base body (2), in particular in a recess (3) of the base body (2).

2. Measurement device (1) according to claim 1,
characterized in that the at least one sensor (5) is elastically mounted in the base body (2), in particular in the recess (3) of the base body (2).

3. Measurement device according to claim 1, characterized in that the level of damping or the elasticity of the mounting are adjustable by the means for pressure regulation (7).

4. Measurement device according to claim 3, characterized in that the damping is adjustable by the means for pressure regulation (7) in dependence of an external force (Fa) acting on the sensor (5), in particular automatically adjustable, in particular for reduction or elimination of an effect of the external force (Fa) on the sensor (5).

5. Measurement device according to claim 3, characterized in that the contact pressure of the at least one sensor (5) is adjustable in a measuring position by the means for pressure regulation (7), in particular automatically adjustable, further in particular in dependence of a or the external force (Fa).

6. Measurement device according to claim 4, characterized in that the measurement device, in particular the means for pressure regulation (7), comprise at least one further sensor for the determination of the external force (Fa) and/or the contact pressure, for example a force sensor, a pressure sensor, a movement sensor or acceleration sensor or a position sensor.

7. Measurement device according to claim 3, characterized in that the measurement device, in particular the means for pressure regulation (7), comprises at least one of the following for the adjusting of the level of damping or the elasticity of the mounting:
servomotor (72),
torsion bar,
camshaft,
spindle (73),
eccentric disk,
solenoid valve or solenoid and plunger,
one or more magnets or electromagnets,
electrode or electrode pair, in particular capacitor,
tension spring,
pressure spring,
membrane,
hydraulic or pneumatic element, such as e.g. a piston (74), a cylinder (75), a pressure chamber or air cushion, a gas spring,
mechanical component made of an electrically deformable material, in particular made of an electroactive elastomer or piezo material.

8. Measurement device (1) according to claim 1, characterized in that the at least one sensor (5) comprises at least one pressure measurement unit.

9. Measurement device (1) according to claim 1, characterized in that the at least one sensor (5) is mounted in a or the recess (3) of the base body (2) by at least one spring element (6), in particular by two, three, four or more springs (6a), e.g. metallic helical springs.

10. Measurement device (1) according to claim 9, characterized in that the at least one spring element (6) is an elastic band and/or an elastomer, in which the at least one sensor (5) is embedded.

11. Measurement device (1) according to claim 1, characterized in that the at least one sensor (5) is plane, bent and/or flexible.

12. Measurement device set comprising at least one measurement device (1) according to claim 9 and at least one further spring element (6), which is configured to replace the available at least one spring element (6) or to mount with it the at least one sensor (5), in particular in series or parallel connection.

13. Measurement system (50) comprising a measurement device (1) according to claim 1 and a computer (51) connected to the measurement device (1) for entering of patient-related information and/or reading of measured vital parameters, wherein the patient-related information comprises in particular age, sex, fitness level, pre-existing conditions such as for example diabetes or COPD, or skin texture, and wherein the computer (51) is arranged either at the measurement device (1) or designed separately, in particular in form of a mobile phone.

14. Method for determining a pressure with which the sensor (5) of a measurement device of a measurement system (50) according to claim 13 lies on the skin of the patient, comprising:
setting the pressure by the computer (51) based on an algorithm saved in the computer (51), in particular an iterative algorithm, optionally further based on at least one patient-related information, again optional further based on at least one value such as movement or acceleration, time, position of the sensor (5) relatively to the standard fixation to the body and gravity.

15. Method for measuring at least one vital parameter comprising:
arrangement of the measurement device (1) of a measurement system (50) according to claim 13 on the patient;
entering of at least one patient-related information in the computer (51) of the measurement system (50) according to claim 13 or retrieving of at least one saved patient-related information from the computer (51) of the measurement system (50) according to claim 13;
adjustment of the pressure with which the sensor (5) of the measurement device lies on the skin based on the at least one patient-related information.

16. Method according to claim 15, further comprising:
carrying out at least one initial measurement of at least one vital parameter for obtaining at least one initial measurement value;
adjustment of the pressure with which the sensor (5) of the measurement device lies on the skin based on the at least one patient-related information and the at least one initial measurement value.

17. Method for checking the setting of the pressure with which the at least one sensor (5) of a measurement device (1) according to claim 1 lies on the skin, comprising:
determining a first measurement value of a vital parameter, while the at least one sensor (5) lies on the skin with a preset pressure.
decreasing the preset pressure of the at least one sensor (5), in particular by lifting the at least one sensor (5) from the skin and/or by adjusting the level of the damping or the elasticity of the mounting of the sensor (5);
determining a second measurement value of the vital parameter, while the at least one sensor (5) lies on the skin with the lowered pressure;
increasing the lowered pressure of the at least one sensor (5), in particular to the preset pressure and/or in particular by lowering the at least one sensor (5) form the skin and/or by adjusting the level of damping or the elasticity of the mounting of the sensor (5);
comparing the first measurement value and the second measurement value.

18. Method for calibrating and/or adjusting the pressure with which the at least one sensor (5) of the measurement device (1) according to claim 1 lies on the skin, comprising:
determining a first measurement value of a vital parameter, while the at least one sensor (5) lies on the skin with a preset pressure.
changing the preset pressure of the at least one sensor (5), in particular decreasing of the preset pressure of the at least one sensor (5), preferably by lifting the at least one sensor (5) from the skin and/or by adjusting the level of damping or the elasticity of the mounting of the sensor (5);
determining a second measurement value of the vital parameter, while the at least one sensor (5) lies on the skin with the changed, or in particular lowered pressure;
comparing the first measurement value and the second measurement value;
adjusting the pressure with which the at least one sensor (5) of the measurement device lies on the skin based on the comparison of the first measurement value and the second measurement value, in particular by lowering the pressure in case of a deviation of the measurement values by 0.2% or more; and/or
calibrating the pressure specified by an algorithm with which the at least one sensor (5) should lie on the skin based on the comparison of the first measurement value and the second measurement value, in particular by lowering the specified pressure in case of a deviation of the measurement values by 0.2% or more.

19. Method for calibrating and/or adjusting the pressure with which the at least one sensor (5) of the measurement device (1) according to claim 1 lies on the skin, comprising:
determining a first measurement value of a vital parameter, while the at least one sensor (5) lies on the skin with a preset pressure.
changing the preset pressure of the at least one sensor (5), in particular decreasing of the preset pressure of the at least one sensor (5), preferably by lifting the at least one sensor (5) from the skin and/or by adjusting the level of damping or the elasticity of the mounting of the sensor (5);
determining a second measurement value of the vital parameter, while the at least one sensor (5) lies on the skin with the changed, or in particular lowered pressure;
changing the changed pressure, in particular by increasing the changed, or lowered pressure of the at least one sensor (5), in particular to the preset pressure or a pressure deviating from it, preferably by lowering the at least one sensor (5) from the skin and/or by adjusting the level of the damping or the elasticity of the mounting of the sensor (5);
determining a third measurement value of the vital parameter, while the at least one sensor (5) lies on the skin with the again changed, or in particular increased pressure;
comparing the first measurement value and the second measurement value and/or comparing the first measurement value and the third measurement value;
adjusting the pressure with which the at least one sensor (5) of the measurement device lies on the skin based on the comparison of the first measurement value and the second measurement value and/or based on the comparison of the first measurement value and the third measurement value, in particular by lowering the pressure in case of a deviation of the measurement values by 0.2% or more; and/or
calibrating the pressure specified by an algorithm with which the at least one sensor (5) should lie on the skin based on the comparison of the first measurement value and the second measurement value and/or based on the comparison of the first measurement value and the third measurement value, in particular by lowering the specified pressure in case of a deviation of the measurement values by 0.2% or more.

20. Use of a measurement device according to claim 1 for determining at least one of the following vital parameters:
blood pressure;
pulse;
blood sugar;
oxygen saturation;
temperature;
skin texture;
moisture.

21. Use according to claim 20 for determining at least one of the following values:
movement or acceleration;
pressure of the sensor on the skin;
time;
position of the sensor (5) relatively to the standard fixation at the body;
gravity.
